SEQUENCE LISTING (2 sequences)

SEQ ID NO: 1 - protein, 18 aa, Artificial Sequence
  Synthetic oligopeptide
SSGGGGSGGGGGGSSRSS

SEQ ID NO: 2 - protein, 7 aa, Artificial Sequence
  Synthetic oligopeptide
GGSSRSS

(19) United States
(12) Patent Application Publication
(10) Pub. No.: US 2017/0114149 A1
(43) Pub. Date: Apr. 27, 2017

(54) METHODS AND COMPOSITIONS FOR IN VIVO NON-COVALENT LINKING

(71) Applicant: Poseida Therapeutics, Inc., San Diego, CA (US)

(72) Inventors: Eric Ostertag, San Diego, CA (US); Tseten Yeshi, Lexington, KY (US); Xianghong Li, Lexington, KY (US)

(21) Appl. No.: 15/317,027

(22) PCT Filed: Jun. 17, 2015
(86) PCT No.: PCT/US2015/036233

§ 371 Date: Dec. 7, 2016

Related U.S. Application Data
(60) Provisional application No. 62/163,568, filed on May 19, 2015.
(60) Provisional application No. 62/013,422, filed on Jun. 17, 2014.

(51) Int. Cl. C07K 16/44; C12N 15/10; C12N 15/64

(52) U.S. Cl. CPC C07K 16/44; C12N 15/64; C12N 15/1055; C07K 2317/622

(57) ABSTRACT

Disclosed are methods of facilitating the interaction of a first and a second component, the method including the use of an antibody fragment and an epitope tag. The antibody fragment may be bound to a first component, while the epitope tag may be bound to a second component. The antibody fragment may have a binding specificity for the epitope tag sufficient to cause an interaction between the antibody fragment and the epitope tag.

### RELATED APPLICATIONS

This application claims the benefit of provisional application U.S. Ser. No. 62/013,422, filed Jun. 17, 2014 and U.S. Ser. No. 62/163,568, filed May 19, 2015, the contents of which are each herein incorporated by reference in their entirety.

### INCORPORATION OF SEQUENCE LISTING

The contents of the text file named “POTH-002/001WO_SeqList.txt,” which was created on Jun. 5, 2015 and is 1 KB in size, are hereby incorporated by reference in their entirety.

### FIELD OF THE DISCLOSURE

The present disclosure relates generally to compositions and methods for site-directed genome modification.

### BACKGROUND

It is a common practice in molecular biology to attach one protein to another with a covalent attachment via a peptide bond. One method of carrying this out is by adding the DNA coding sequence of one protein downstream of the coding sequence for a second protein, such that the two sequences will be translated as a single polypeptide. One problem with this strategy, however, is that a protein can only be conveniently linked to another protein at the protein's amino terminus (N-terminus) or carboxy terminus (C-terminus). Unfortunately, combining proteins in this manner will oftentimes cause one or both of the proteins to fold incorrectly. Even if folded correctly, it is not uncommon for one or both of the fused proteins to be non-functional due to one protein physically interfering with normal functioning of the other protein. These problems may be alleviated to some extent by the use of a flexible linker encoded between the two polypeptides. However, many protein fusions may still not be functional despite the use of a linker sequence, such that problems with this technique remain.

A second problem with the above-described fusion protein strategies is that the process creates one large protein that is much larger than either of the individual single proteins. This too can compromise function or the ability of the fused protein to access the desired locations in vivo. Further, it is often desirable to instead deliver DNA that encodes for the desired fused protein into cells via viral delivery methods. However, viral delivery methods are limited by the amount of DNA that they can contain. DNA encoding large fusion proteins may not fit in viral delivery vehicles (such as, for example, Adeno Associated Virus (AAV)), thereby limiting the utility of this method.

A further drawback of the above described methods is the limitations that occur when only transient interactions are desired. It is sometimes desirable or advantageous to allow functional associations of two or more proteins at different time points. This can be due, for example, to the multiple functions of a given protein. Transposase proteins are one such type of protein. Transposase proteins perform several important steps, including transposon recognition, cleavage of DNA to excise a transposon, movement of transposon sequence to a new genomic location, recognition of a new target site and cleavage of DNA to integrate the transposon at a new locus. In such cases, it may be desirable to direct a transposase to a particular site in the genome by adding a heterologous protein with site-specific DNA binding. However, this protein would only be required during the target site recognition step and its presence at earlier stages may actually be detrimental to the other steps, such that a fusion protein could not be practically applied. As such, there is a need in the art for methods that address one or more of the above-described problems in the art. The instant disclosure seeks to address one or more of the aforementioned deficiencies in the art.

### BRIEF SUMMARY

Disclosed are methods of facilitating the interaction of a first and a second component, the method including the use of an antibody fragment and an epitope tag. The antibody fragment may be bound to a first component, while the epitope tag may be bound to a second component. The antibody fragment may have a binding specificity for the epitope tag sufficient to cause an interaction between the antibody fragment and the epitope tag.

The disclosure provides a method of facilitating the interaction of a first and a second component, comprising providing an antibody fragment bound to a first component; and providing an epitope tag bound to a second component; wherein said antibody fragment comprises binding specificity for said epitope tag sufficient to cause an interaction between said antibody fragment and said epitope tag. In certain embodiments of this method, the antibody fragment and said epitope tag transiently interact. This transient interaction may occur in the interior of a cell.

Antibody fragments of the disclosure may comprise a single chain variable fragment (ScFv). Alternatively, or in addition, antibody fragments of the disclosure may comprise a single chain variable fragment (ScFv), a single domain antibody (sdAb), a domain antibody, a SMIP, or a combination thereof.

In certain embodiments of the methods of the disclosure, the first and second component comprise an epitope tag covalently attached to target protein and a ScFv covalently attached to a signal.

Antibody fragments of the disclosure may comprise a single domain antibody (sdAb).

First components of the disclosure may be a protein, a small molecule, a fluorophore, a signal peptide, a nanoparticle, a cellular component, or any combination thereof. In certain embodiments, the first component comprises an effector molecule. Exemplary effector molecule include, but are not limited to, a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, or any combination thereof. Effector molecules of the disclosure may be capable of modifying gene expression.

Second components of the disclosure may comprise an effector protein. In certain embodiments in which the second component is an effector protein, exemplary effector proteins include, but are not limited to, a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, or a combination thereof. Effector molecules of the disclosure may be capable of modifying gene expression.

In certain embodiments of the methods of the disclosure, the effector molecule is a nuclease. The effector molecule may be BfilI. The effector molecule may be BmrI. The effector molecule may be Clo051. The effector molecule may be FokI.

Second components of the disclosure may include a protein, a small molecule, a fluorophore, a signal peptide, a nanoparticle, a cellular component, or any combination thereof. In certain embodiments of the methods of the disclosure, the second component comprises a protein capable of modifying gene expression. The second component may comprise a protein that modifies DNA.

The disclosure provides a kit comprising an antibody fragment and an epitope tag; wherein said antibody fragment is bound to a first component; wherein said epitope tag is bound to said second component; and wherein said antibody fragment comprises binding specificity for said epitope tag sufficient to cause an interaction between said antibody fragment and said epitope tag. In certain embodiments of the kits of the disclosure, the first and the second component are a protein, a small molecule, a fluorophore, a signal peptide, a nanoparticle, a cellular component, or any combination thereof.

The disclosure provides a method of facilitating the interaction of a first and a second component, comprising providing a scaffold protein bound to a first component; and providing a corresponding binding site bound to a second component; wherein said scaffold protein specifically binds to the corresponding binding site to cause an interaction between the scaffold protein and the corresponding binding site. In certain embodiments of the methods of the disclosure, the scaffold protein and the corresponding binding site transiently interact. The transient interaction may occur in the interior of a cell.

In certain embodiments of the methods of the disclosure, scaffold proteins may comprise an antibody mimetic.

In certain embodiments of the methods of the disclosure, scaffold proteins may comprise a single chain variable fragment (ScFv), a domain antibody, a nanobody, a SMIP, or any combination thereof. Domain antibodies may comprise or consist of a single domain antibody (sdAb).

In certain embodiments of the methods of the disclosure, scaffold proteins may comprise an affibody, an affilin, an affimer, an affitin, an alphabody, an anticalin, an avimer, a DARPin, a Fynomer, a Kunitz domain or a Kunitz domain peptide, a monobody, or any combination thereof.

Scaffold proteins of the disclosure may be covalently bound to a first component.

First components of the disclosure may comprise a protein, a small molecule, a fluorophore, a signal peptide, a nanoparticle, a cellular component, or any combination thereof. Moreover, first components of the disclosure may comprise an effector molecule. Exemplary effector molecules include, but are not limited to, a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, or any combination thereof. Effector molecules may be capable of modifying gene expression.

Second components of the disclosure may comprise a protein, a small molecule, a fluorophore, a signal peptide, a nanoparticle, a cellular component, or any combination thereof. In certain embodiments in which the second component comprises a protein, the protein may be capable of modifying gene expression and/or modify DNA.

Second components of the disclosure may comprise an effector protein. In certain embodiments of the methods of the disclosure, when the second component is an effector protein, exemplary effector proteins include, but are not limited to, a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, or any combination thereof. Effector molecules may be capable of modifying gene expression.

In certain embodiments of the methods of the disclosure, the effector molecule is a nuclease. The effector molecule may be BfilI. The effector molecule may be BmrI. The effector molecule may be Clo051. The effector molecule may be FokI.

The disclosure provides a kit comprising a scaffold protein and a corresponding binding site; wherein the scaffold protein is bound to a first component; wherein the corresponding binding site is bound to the second component; and wherein the scaffold protein specifically binds to the corresponding binding site to cause an interaction between the scaffold protein and the corresponding binding site. In certain embodiments of the kits of the disclosure, the first and the second component may be a protein, a small molecule, a fluorophore, a signal peptide, a nanoparticle, a cellular component, or any combination thereof.

According to certain embodiments of the methods of the disclosure, the first and the second component comprise a first protein and a second protein, respectively.

According to certain embodiments of the methods of the disclosure, the first and the second component comprise a DNA binding protein and an effector protein, respectively, wherein the interaction of the first and the second component results in a change in gene expression or a modification of DNA.

According to certain embodiments of the methods of the disclosure, the first and the second component comprise a fluorophore and a protein, respectively, wherein the interaction of the first and the second component permits real-time monitoring of protein expression and subcellular localization.

According to certain embodiments of the methods of the disclosure, the first and the second component comprise a first and second small molecule, respectively, and are capable of interacting to activate a prodrug.

The disclosure provides a method for modifying a genome of an organism comprising the steps of providing an antibody fragment bound to a first component, wherein the first component is a DNA binding molecule, and providing an epitope tag bound to a second component, wherein the second component is an effector molecule capable of modifying gene expression, wherein said antibody fragment comprises binding specificity for said epitope tag sufficient to cause an interaction between said antibody fragment and said epitope tag. According to this method, an interaction between the DNA binding molecule and the effector molecule results in a change in gene expression or a modification of DNA. In certain embodiments, the DNA binding molecule is a DNA, RNA, or protein. DNA binding molecules of the disclosure may specifically target a locus or loci within a genomic sequence. In certain embodiments, the effector molecule is an endonuclease. According to this method, a genome may be modified when one or more genomic sequences or base pairs are separated by an endonuclease and/or when one or more genomic sequences or base pairs are deleted, inserted, substituted, inverted, or relocated. Moreover, the disclosure provides a cell comprising a genomic sequence or base pair modified by a method of the disclosure. Cells modified by the methods of the disclosure may comprise, for example, a deletion, an insertion, a substitution, an inversion, or a relocation of a genomic sequence or base pair of the genome. Cells modified according to the methods of the disclosure may comprise, for example, an exogenous, artificial, or heterologous sequence that does not naturally-occur within the genome of that cell. The cell may be modified according to a method of the disclosure in vivo, ex vivo, or in vitro. In certain embodiments, the cell is neither a human cell nor a human embryonic cell.

### BRIEF DESCRIPTION OF THE DRAWINGS

FIG. 1 is a schematic diagram depicting the method of phage display to generate scFv against piggyBac. Rabbits are immunized with PB transposase protein (PBase) for expanding relevant B cells. Variable regions from heavy and light chain (VH and VL) genes are amplified from cDNA by PCR to form fusion products containing an 18 amino acid linker (L). Phagemid are produced, panned against PBase, amplified in E. coli, and repeated once or twice. The resulting phagemid DNA library is cloned into the pLVX-IRES-ZsGreen vector containing the E2c PZF with a linker sequence. An E2c-scFv N-terminal fusion library is then produced in Lentivirus.

### DETAILED DESCRIPTION

### Definitions

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within one or more than one standard deviations, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within five-fold, or within two-fold, of a value. Where particular values are described in the application and claims, unless otherwise stated the term “about” meaning within an acceptable error range for the particular value should be assumed.

“Binding” refers to a specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is specific.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

As used herein, the term “comprising” is intended to mean that the compositions and methods include the recited elements, but do not exclude others. “Consisting essentially of when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Embodiments defined by each of these transition terms are within the scope of this invention.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to one embodiment hereof, the term “antibody hereof” in its broadest sense also covers such analogs. Generally, in such analogs, one or more amino acid residues may have been replaced, deleted and/or added, compared to the antibodies hereof as defined herein.

“Antibody fragment,” and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). The term further includes single domain antibodies (“sdAB”) refers to antibody fragments having a single monomeric variable antibody domain, (for example, from camelids). Such antibody fragment types will be readily understood by a person having ordinary skill in the art.

As used herein, the term “epitope tag”, or otherwise “affinity tag”, refers to a short amino acid sequence or peptide enabling a specific interaction with a protein or a ligand.

As used herein, “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

As used herein, the term “operatively linked” or its equivalents (e.g., “linked operatively”) means two or more molecules are positioned with respect to each other such that they are capable of interacting to affect a function attributable to one or both molecules or a combination thereof.

The term “scFv” refers to a single-chain variable fragment. scFv is a fusion protein of the variable regions of the heavy (VH) and light chains (VL) of immunoglobulins, connected with a linker peptide. The linker peptide can be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g., Protein G) used to purify antibodies. The term further includes a scFv that is an intrabody, an antibody that is stable in the cytoplasm of the cell, and which may bind to an intracellular protein.

As used herein, the term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In certain embodiments, a specific binding interaction will discriminate between desirable and undesirable antigens in a sample, in some embodiments more than about ten- to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

The disclosure provides compositions and methods for linking proteins non-covalently after they have been produced in the cell. In one aspect, the disclosed methods allow for transient (temporary) interactions of two or more molecules, for a period of time sufficient to allow for a desired effect. As a result of the ability of two or more molecules to associate and dissociate, a functional association may be enabled only when critical.

The disclosure provides a method of facilitating the interaction of a first and a second component.

Methods of the disclosure may comprise the steps of providing an antibody fragment bound to a first component, and providing an epitope tag bound to a second component, wherein the antibody fragment may comprise binding specificity for said epitope tag sufficient to cause an interaction between the antibody fragment and the epitope tag. In one aspect, the antibody fragment and epitope tag are selected such that the antibody fragment and epitope tag transiently interact. As contemplated herein, the interaction occurs in the interior of a cell.

Antibody fragments of the disclosure may be covalently bound to the first component. The antibody fragment may be any antibody fragment understood to one of ordinary skill in the art. In one aspect, the antibody fragment may comprise a single chain variable fragment (ScFv). In one aspect, the antibody fragment may comprise a single domain antibody (sdAb). For example, the method may utilize an epitope tag covalently attached to a target protein and a ScFv covalently attached to a second protein.

Methods of the disclosure may comprise the steps of providing an antibody mimetic or a scaffold protein bound to a first component, and providing a corresponding binding site of the antibody mimetic or scaffold protein bound to a second component, wherein the antibody mimetic or a scaffold protein may specifically bind to the corresponding binding site of the antibody mimetic or scaffold protein and result in an interaction between the antibody mimetic or a scaffold protein and the corresponding binding site. In one aspect, the antibody mimetic or a scaffold protein and corresponding binding site are selected such that the antibody mimetic or a scaffold protein and corresponding binding site transiently interact. As contemplated herein, the interaction occurs in the interior of a cell.

Antibody mimetics of the disclosure may be covalently bound to the first component. As used herein, the term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g. resistance to enzymatic degradation), and lower production costs. Exemplary antibody mimetics include, but are not limited to, an affibody, an afflilin, an affimer, an affitin, an alphabody, an anticalin, and avimer (also known as avidity multimer), a DARPin (Designed Ankyrin Repeat Protein), a Fynomer, a Kunitz domain peptide, and a monobody.

Affibody molecules of the disclosure comprise a protein scaffold comprising or consisting of one or more alpha helix without any disulfide bridges. Preferably, affibody molecules of the disclosure comprise or consist of three alpha helices. For example, an affibody molecule of the disclosure may comprise an immunoglobulin binding domain. An affibody molecule of the disclosure may comprise the Z domain of protein A.

Affilin molecules of the disclosure comprise a protein scaffold produced by modification of exposed amino acids of, for example, either gamma-B crystallin or ubiquitin. Affilin molecules functionally mimic an antibody's affinity to antigen, but do not structurally mimic an antibody. In any protein scaffold used to make an affilin, those amino acids that are accessible to solvent or possible binding partners in a properly-folded protein molecule are considered exposed amino acids. Any one or more of these exposed amino acids may be modified to specifically bind to a target sequence or antigen.

Affimer molecules of the disclosure comprise a protein scaffold comprising a highly stable protein engineered to display peptide loops that provide a high affinity binding site for a specific target sequence. Exemplary affimer molecules of the disclosure comprise a protein scaffold based upon a cystatin protein or tertiary structure thereof. Exemplary affimer molecules of the disclosure may share a common tertiary structure of comprising an alpha-helix lying on top of an anti-parallel beta-sheet.

Affitin molecules of the disclosure comprise an artificial protein scaffold, the structure of which may be derived, for example, from a DNA binding protein (e.g. the DNA binding protein Sac7d). Affitins of the disclosure selectively bind a target sequence, which may be the entirety or part of an antigen. Exemplary affitins of the disclosure are manufactured by randomizing one or more amino acid sequences on the binding surface of a DNA binding protein and subjecting the resultant protein to ribosome display and selection. Target sequences of affitins of the disclosure may be found, for example, in the genome or on the surface of a peptide, protein, virus, or bacteria. In certain embodiments of the disclosure, an affitin molecule may be used as a specific inhibitor of an enzyme. Affitin molecules of the disclosure may include heat-resistant proteins or derivatives thereof.

Alphabody molecules of the disclosure may also be referred to as Cell-Penetrating Alphabodies (CPAB). Alphabody molecules of the disclosure comprise small proteins (typically of less than 10 kDa) that bind to a variety of target sequences (including antigens). Alphabody molecules are capable of reaching and binding to intracellular target sequences. Structurally, alphabody molecules of the disclosure comprise an artificial sequence forming single chain alpha helix (similar to naturally occurring coiled-coil structures). Alphabody molecules of the disclosure may comprise a protein scaffold comprising one or more amino acids that are modified to specifically bind target proteins. Regardless of the binding specificity of the molecule, alphabody molecules of the disclosure maintain correct folding and thermostability.

Anticalin molecules of the disclosure comprise artificial proteins that bind to target sequences or sites in either proteins or small molecules. Anticalin molecules of the disclosure may comprise an artificial protein derived from a human lipocalin. Anticalin molecules of the disclosure may be used in place of, for example, monoclonal antibodies or fragments thereof. Anticalin molecules may demonstrate superior tissue penetration and thermostability than monoclonal antibodies or fragments thereof. Exemplary anticalin molecules of the disclosure may comprise about 180 amino acids, having a mass of approximately 20 kDa. Structurally, anticalin molecules of the disclosure comprise a barrel structure comprising antiparallel beta-strands pairwise connected by loops and an attached alpha helix. In preferred embodiments, anticalin molecules of the disclosure comprise a barrel structure comprising eight antiparallel beta-strands pairwise connected by loops and an attached alpha helix.

Avimer molecules of the disclosure comprise an artificial protein that specifically binds to a target sequence (which may also be an antigen). Avimers of the disclosure may recognize multiple binding sites within the same target or within distinct targets. When an avimer of the disclosure recognize more than one target, the avimer mimics function of a bi-specific antibody. The artificial protein avimer may comprise two or more peptide sequences of approximately 30-35 amino acids each. These peptides may be connected via one or more linker peptides. Amino acid sequences of one or more of the peptides of the avimer may be derived from an A domain of a membrane receptor. Avimers have a rigid structure that may optionally comprise disulfide bonds and/or calcium. Avimers of the disclosure may demonstrate greater heat stability compared to an antibody.

DARPins (Designed Ankyrin Repeat Proteins) of the disclosure comprise genetically-engineered, recombinant, or chimeric proteins having high specificity and high affinity for a target sequence. In certain embodiments, DARPins of the disclosure are derived from ankyrin proteins and, optionally, comprise at least three repeat motifs (also referred to as repetitive structural units) of the ankyrin protein Ankyrin proteins mediate high-affinity protein-protein interactions. DARPins of the disclosure comprise a large target interaction surface.

Fynomers of the disclosure comprise small binding proteins (about 7 kDa) derived from the human Fyn SH3 domain and engineered to bind to target sequences and molecules with equal affinity and equal specificity as an antibody.

Kunitz domain peptides of the disclosure comprise a protein scaffold comprising a Kunitz domain. Kunitz domains comprise an active site for inhibiting protease activity. Structurally, Kunitz domains of the disclosure comprise a disulfide-rich alpha+beta fold. This structure is exemplified by the bovine pancreatic trypsin inhibitor. Kunitz domain peptides recognize specific protein structures and serve as competitive protease inhibitors. Kunitz domains of the disclosure may comprise Ecallantide (derived from a human lipoprotein-associated coagulation inhibitor (LACI)).

Monobodies of the disclosure are small proteins (comprising about 94 amino acids and having a mass of about 10 kDa) comparable in size to a single chain antibody. These genetically engineered proteins specifically bind target sequences including antigens. Monobodies of the disclosure may specifically target one or more distinct proteins or target sequences. In preferred embodiments, monobodies of the disclosure comprise a protein scaffold mimicking the structure of human fibronectin, and more preferably, mimicking the structure of the tenth extracellular type III domain of fibronectin. The tenth extracellular type III domain of fibronectin, as well as a monobody mimetic thereof, contains seven beta sheets forming a barrel and three exposed loops on each side corresponding to the three complementarity determining regions (CDRs) of an antibody. In contrast to the structure of the variable domain of an antibody, a monobody lacks any binding site for metal ions as well as a central disulfide bond. Multispecific monobodies may be optimized by modifying the loops BC and FG. Monobodies of the disclosure may comprise an adnectin.

Scaffold proteins of the disclosure may be covalently bound to the first component. Scaffold proteins of the disclosure include, for example, antibody mimetics of the disclosure. Scaffold proteins of the disclosure further include, for example, small modular immunopharmaceutical (SMIP) molecules, a domain antibody, and a nanobody.

SMIP molecules of the disclosure are artificial proteins comprising one or more sequences or portions of an immunoglobulin (antibody) that are monospecific for a target sequence or antigen. SMIPs of the disclosure may substitute for the use of a monoclonal antibody. Structurally, SMIPs are single chain proteins comprising a binding region, a hinge region (i.e. a connector), and an effector domain. The binding region of a SMIP may comprise a modified single-chain variable fragment (scFv). SMIPs may be produced from genetically-modified cells as dimers.

Domain antibodies of the disclosure comprise a single monomeric variable antibody domain (i.e. either heavy or light variable domain). Domain antibodies of the disclosure demonstrate the same antigen specificity as a whole and intact antibody. Domain antibodies of the disclosure may be manufactured, at least in part, by immunization of dromedaries, camels, llamas, alpacas or sharks with the desired antigen and subsequent isolation of the mRNA coding for heavy-chain antibodies.

Nanobodies of the disclosure comprise a VHH single domain antibody. Nanobodies of the disclosure may comprise single domain antibodies of the disclosure.

The various components contemplated herein may take a variety of different forms and may be readily appreciated by one of ordinary skill in the art. For example, the first or second component may be selected from a protein, a small molecule, a fluorophore, a signal peptide, a nanoparticle, a cellular component, and combinations thereof. In other aspects, the first or second component may comprise an effector molecule. The effector molecule is generally a molecule capable of a predetermined effect at said specific loci. In one aspect, the effector molecule may be selected from a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, and combinations thereof. In one aspect, the effector molecule may be FokI.

In one aspect, the effector molecule may comprise a nuclease. Suitable nucleases include, but are not limited to, restriction endonucleases, homing endonucleases, 51 Nuclease, mung bean nuclease, pancreatic DNase I, micrococcal nuclease, yeast HO endonuclease, or any combination thereof. In one aspect, the effector molecule may comprise a Type IIS restriction endonuclease. For example, in some aspects, the effector molecule may comprise an endonuclease selected from AciI, MnlI, AlwI, BbvI, BccI, BceAI, BsmAI, BsmFI, BspCNI, BsrI, BtsCI, HgaI, HphI, HpyAV, MbolI, MylI, PleI, SfaNI, AcuI, BciVI, BfuAI, BmgBI, BmrI, BpmI, BpuEI, BsaI, BseRI, BsgI, BsmI, BspMI, BsrBI, BsrBI, BsrDI, BtgZI, BtsI, EarI, EciI, MmeI, NmeAIII, BbvCI, Bpu10I, BspQI, SapI, BaeI, BsaXI, CspCI, FokI BfiI, MboII, Acc36I, FokI, BfiI, MboII, BspMI, BsgI, BpmI, Acc36I, or Clo051. In other aspects, the effector molecule may comprise a PB transposase (PBase).

The first or second component may comprise a protein capable of modifying gene expression. In other aspects, the first or second component may comprise a protein capable of modifying DNA. In yet further aspects, the first or second component may comprise a first protein and a second protein.

In one aspect, the first and second component may comprise a DNA binding protein and an effector protein, respectively, wherein an interaction of the first and second component results in a change in gene expression or a modification of DNA. In other aspects, the first and second component may comprise a fluorophore and a protein, respectively, wherein an interaction of the first and second component permits real-time monitoring of protein expression and subcellular localization. In yet further aspects, the first and second component may comprise, respectively, a first and second small molecule wherein an interaction of the first and second component activates a prodrug.

Also disclosed are kits that may comprise an antibody fragment bound to a first component and an epitope tag bound to a second component, wherein the antibody fragment comprises binding specificity for the epitope tag sufficient to cause an interaction between said antibody fragment and said epitope tag. The kit may further comprise instructions for use. The antibody fragment, epitope tag, and first and second components may be as described above.

The disclosed systems and compositions may be used in the manufacture of medicaments and for the treatment of humans and other animals by administration in accordance with conventional procedures, such as an active ingredient in pharmaceutical compositions.

In one aspect, a detectable label may be used. One or more detectable labels or other signal-generating groups or moieties, depending on the intended use of the labeled antibody, may be used in the disclosed methods. Suitable labels and techniques for attaching, using and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metal chelates or metallic cations or other metals or metallic cations that are particularly suited for use in vivo, in vitro or in situ imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy. Such labeled antibodies hereof may, for example, be used for in vitro, in vivo or in situ assays (including immunoassays known per se such as ELISA, RIA, EIA and other “sandwich assays,” etc.) as well as in vivo imaging purposes, depending on the choice of the specific label. As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link the antibody hereof to another protein, polypeptide or chemical compound that is bound to the other half of the binding pair, i.e., through formation of the binding pair. For example, an antibody hereof may be conjugated to biotin, and linked to another protein, polypeptide, compound or carrier conjugated to avidin or streptavidin. For example, such a conjugated antibody may be used as a reporter, for example, in a system where a detectable signal-producing agent is conjugated to avidin or streptavidin.

The above-described methods can be used for a variety of different applications. Non-limiting examples include tagging two proteins such that they come together in vivo (for example, an epitope tag covalently attached to one protein and antibody fragment covalently attached to the other); linking a DNA binding protein to an effector protein to affect gene expression or modify DNA (e.g. dCas9 linked to Fok I); linking a fluorophore to a protein to allow real-time monitoring of protein expression and subcellular localization (e.g. EGFP linked to a target protein); bringing two proteins in close proximity in the cell for any desired reason (e.g. Fluorescence Resonance Energy Transfer); tagging a protein with a signal peptide or post-translational modification (epitope tag covalently attached to target protein and ScFv covalently attached to signal or vice versa); adding an E3 ligase to target a protein for degradation via the ubiquitination pathway; adding a target peptide or signal peptide to move a protein to a new subcellular localization (e.g. endoplasmic reticulum retention signal, nuclear localization signal, nucleolar localization signal, mitochondrial targeting signal, peroxisomal targeting signal, secretory pathway signals; labelling a protein with a nanoparticle (e.g. for fluorescence imaging); labelling a protein with a small molecule (e.g. using a protein to carry or locally concentrate a small molecule therapeutic); attaching a small molecule to a nanoparticle (e.g. for drug delivery or to concentrate a small molecule therapeutic); bringing two small molecules or two nanoparticles together (e.g. to activate a prodrug). These and many other applications are encompassed by the claims as set forth herein, and are not intended to limit the scope of the invention.

### Examples

Phage display is used to identify a scFv antibody against a FLAG affinity tag that provides an optimal linkage. A large diversity in scFv affinity is obtained by limiting the stringency of the affinity selection process. This diversity may represent a key advantage of a PhD approach for identifying a successful linkage between a FLAG affinity tag and a scFv with affinity for the FLAG tag. In some instances, a single-chain variable fragment (scFv) antibody with a faster off-rate may provide permissive “breathing” of a scFv-FLAG complex. A near-exhaustive search among scFv antibodies allows one to select from among a large diversity of possible conformations of scFv-FLAG affinity tag complexes. A PhD strategy may create such diversity through the generation of unique monovalent scFvs against the FLAG epitope.

A non-covalent linkage method, such as that achieved through the use of a scFv antibody employs a protein fused to a scFv that provides a reversible association between a FLAG affinity tag and the scFv, which may circumvent any permanent interference with the target protein that may occur when it is subjected to covalent linkage.

Immunization for Producing Anti-FLAG Antibodies.

An antibody library is produced from immunized rabbits as is well known in the art. Six New Zealand White rabbits are immunized each with 200 pg of a FLAG affinity tag peptide sequence plus adjuvant, and serum is collected six weeks after immunization for determining antibody titers. Titers are determined by ELISAs on immobilized FLAG affinity tag and the animals with the highest titers (at least 1:1000) are sacrificed for isolating the spleen and bone marrow. If rabbits do not produce sufficient titers, a naïve library from embryonic rabbit tissue is used. This provides an unbiased collection of un-rearranged heavy and light chain genes. Total RNA is extracted from tissues using Trizol (Invitrogen), and cDNA synthesis is performed with the iScript cDNA synthesis kit (BioRad).

Generating scFv Gene Fusions.

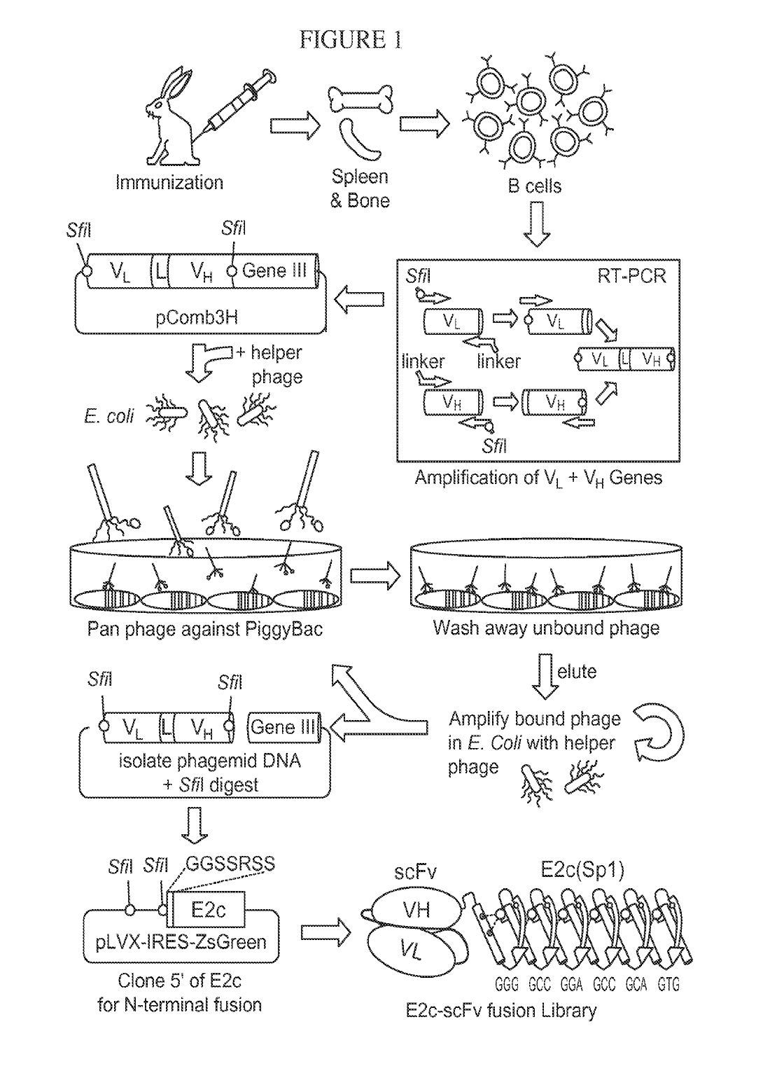

To isolate expressed variable regions of heavy and light chain genes from rabbit, several primers are used. Eight primers are used for kappa and lambda light chain amplification and five primers are used for heavy chain gene amplification. Primers also contain the coding sequence for an 18 amino acid linker sequence (SSGGGGSGGGGGGSSRSS) (SEQ ID NO: 1), which links the variable regions of the heavy and light chains (VH and VL). This longer linker sequence provides better stability of monomeric forms of scFv fragments. The PCR products of the VH and VL genes overlap in this linker region and can then be assembled by overlap-extension (OLE) PCR (FIG. 1). PCR products are then digested with Sfil, ligated with Sfil-digested pComb3H, and DNA will then be size-selected by gel electrophoresis. This plasmid enables phagemid display of an scFv fused to the pill coat protein. About 5 molecules of pill phage coat protein is present on each phage particle. The pComb3H plasmid expresses the scFv-plll fusion at a level such that about one or two molecules are integrated with wild-type pill (which is provided by helper phage). Since up to 1012 phage particles can be generated in a single preparation, a very large number of scFvs can thus be screened. In PhD the scFv coding sequence is always linked to the phage particle displaying the protein, so subsequent DNA sub-cloning is conveniently achieved.

Producing and Screening the Phage Library.

Ligated plasmid DNA (50 to 100 ng) is electroporated into ER2538 E. coli (New England Biolabs). E. coli will then be recovered by shaking for 1 hour at 37° C. in 5 mLs of SOC. Phage is produced with the VCSM13 helper phage, which has a defective origin of replication. Phage particles will be precipitated with PEG-8000 and then isolated by further centrifugation. This phage prep is the primary library, and will be affinity selected by “panning” Double recognition panning is performed in which the phage elution is re-incubated with the immobilized antigen, washed, and eluted again. This helps eliminate non-specific phage. To test each round of selection, phage pools are assayed by ELISAs for affinity to the PB antigen. PB or BSA are coated to 96-well plates, incubated with phage, and then incubated with a horseradish peroxidase (HRP) conjugated anti-M13 antibody, which recognizes the M13 phage coat protein. An increasing ELISA titer indicates successful affinity selection of each phage pool.

Transferring the scFv Library into a Lentiviral Vector, and Expansion in E. coli.

Phagemid DNA is isolated from bacteria after the 2nd (R2) and 3rd (R3) rounds of panning by infecting E. coli with each phage pool, selecting with carbenicillin, followed by standard plasmid preparation. Plasmid DNA is digested with Sfil to liberate the scFv coding sequence, and ligated upstream of the E2c coding sequence within the pLVX-IRES-ZsGreen1 (Clontech) vector. The E2c coding sequence also has a short linker sequence (GGSSRSS) (SEQ ID NO: 2) and creates a fusion of the scFv library to the N-terminal portion of E2c. The two ensuing plasmid libraries (R2 and R3) will then be prepared as in Aim 2, for production of two lentivirus libraries.

Lentivirus Library Production.

For production of lentivirus particles, the Lenti-X HT Packaging System (Clontech) is used, which produces viral titers as high as 5×10⁸ infectious units per mL. Virus is produced according to the manufacturer's specifications. Viral supernatants are titered on HepG2 and Huh7 cells, followed by FACS fluorescence produced by the ZsGreen1 reporter to count transduced cells.

In another aspect, a method for screening for scFvs is disclosed. In this aspect, scFvs that are stable in the cytoplasm may be identified by forming a fusion protein between the scFv and EGFP and expressing in a surrogate mammalian cell line.

The practice of the present invention will employ, unless otherwise indicated, conventional techniques of molecular biology (including recombinant techniques), microbiology, cell biology and biochemistry, which are within the skill of the art.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of facilitating the interaction of a first and a second component, comprising
a. providing an antibody fragment bound to a first component; and
b. providing an epitope tag bound to a second component;
wherein said antibody fragment comprises binding specificity for said epitope tag sufficient to cause a interaction between said antibody fragment and said epitope tag.

2. The method of claim 1 wherein said antibody fragment and said epitope tag transiently interact.

3. The method of claim 1 wherein said antibody fragment and said epitope tag transiently interact, wherein said transient interaction occurs in the interior of a cell.

4. The method of claim 1 wherein said antibody fragment is covalently bound to said first component.

5. The method of claim 1 wherein said antibody fragment comprises a single chain variable fragment (ScFv).

6. The method of claim 1 wherein said antibody fragment comprises a single chain variable fragment (ScFv), a single domain antibody (sdAb), a domain antibody, a SMIP, or a combination thereof.

7. The method of claim 1 wherein said first and second component comprise an epitope tag covalently attached to target protein and a ScFv covalently attached to a signal.

8. The method of claim 1 wherein said first component is selected from a protein, a small molecule, a fluorophore, a signal peptide, a nanoparticle, a cellular component, and combinations thereof.

9. The method of claim 1 wherein said first component comprises an effector molecule.

10. The method of claim 1 wherein said first component comprises an effector molecule selected from a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, and combinations thereof.

11. The method of claim 1 wherein said first component comprises an effector molecule capable of modifying gene expression.

12. The method of claim 1 wherein said second component comprises an effector protein.

13. The method of claim 1 wherein said second component comprises an effector protein selected from a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, and combinations thereof.

14. The method of claim 1 wherein said first component comprises an effector molecule protein capable of modifying gene expression.

15. The method of claim 1 wherein said second component is selected from a protein, a small molecule, a fluorophore, a signal peptide, a nanoparticle, a cellular component, and combinations thereof.

16. The method of claim 1 wherein said second component comprises a protein capable of modifying gene expression.

17. The method of claim 1 wherein said second component comprises a protein, wherein said protein modifies DNA.

18. The method of claim 1 wherein said first and second component comprise a first protein and a second protein.

19. The method of claim 1 wherein said first and second component comprise a DNA binding protein and an effector protein, wherein said interaction results in a change in gene expression or a modification of DNA.

20. The method of claim 1 wherein said first and second component comprise a fluorophore and a protein, wherein said interaction permits real-time monitoring of protein expression and subcellular localization.

21. The method of claim 1 wherein said first and second component comprise a first and second small molecule capable of interacting to activate a prodrug.

22. A kit comprising an antibody fragment and an epitope tag;
wherein said antibody fragment is bound to a first component;
wherein said epitope tag is bound to said second component; and
wherein said antibody fragment comprises binding specificity for said epitope tag sufficient to cause an interaction between said antibody fragment and said epitope tag.

23. The kit of claim 22 wherein said first and second component are selected from a protein, a small molecule, a fluorophore, a signal peptide, a nanoparticle, a cellular component, and combinations thereof.

24. A method of facilitating the interaction of a first and a second component, comprising
a. providing a scaffold protein bound to a first component; and
b. providing a corresponding binding site bound to a second component;
wherein said scaffold protein specifically binds to the corresponding binding site to cause an interaction between the scaffold protein and the corresponding binding site.

25. The method of claim 24 wherein the scaffold protein is an antibody mimetic.

26. The method of claim 24 wherein the scaffold protein and the corresponding binding site transiently interact.

27. The method of claim 26 wherein the transient interaction occurs in the interior of a cell.

28. The method of claim 24 wherein the scaffold protein is covalently bound to said first component.

29. The method of claim 1 wherein the scaffold protein comprises an affibody, an affilin, an affimer, an affitin, an alphabody, an anticalin, an avimer, a DARPin, a Fynomer, a Kunitz domain or a Kunitz domain peptide, a monobody, or a combination thereof.

30. The method of claim 24 wherein the first component is a protein, a small molecule, a fluorophore, a signal peptide, a nanoparticle, a cellular component, or a combination thereof.

31. The method of claim 24 wherein the first component comprises an effector molecule.

32. The method of claim 31 wherein the effector molecule is a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, or a combination thereof.

33. The method of claim 24 wherein said first component comprises an effector molecule capable of modifying gene expression.

34. The method of claim 24 wherein said second component comprises an effector protein.

35. The method of claim 24 wherein said second component comprises an effector protein selected from a transcription factor (activator or repressor), chromatin remodeling factor, exonuclease, endonuclease, transposase, methytransferase, demethylase, acetyltransferase, deacetylase, kinase, phosphatase, integrase, recombinase, ligase, topoisomerase, gyrase, helicase, fluorophore, and combinations thereof.

36. The method of claim 24 wherein said first component comprises an effector molecule protein capable of modifying gene expression.

37. The method of claim 24 wherein said second component is selected from a protein, a small molecule, a fluorophore, a signal peptide, a nanoparticle, a cellular component, and combinations thereof.

38. The method of claim 24 wherein said second component comprises a protein capable of modifying gene expression.

39. The method of claim 24 wherein said second component comprises a protein, wherein said protein modifies DNA.

40. The method of claim 24 wherein said first and second component comprise a first protein and a second protein.

41. The method of claim 24 wherein said first and second component comprise a DNA binding protein and an effector protein, wherein said interaction results in a change in gene expression or a modification of DNA.

42. The method of claim 24 wherein said first and second component comprise a fluorophore and a protein, wherein said interaction permits real-time monitoring of protein expression and subcellular localization.

43. The method of claim 24 wherein said first and second component comprise a first and second small molecule capable of interacting to activate a prodrug.

44. A kit comprising a scaffold protein and a corresponding binding site;
wherein the scaffold protein is bound to a first component;
wherein the corresponding binding site is bound to said second component; and
wherein the scaffold protein specifically binds to the corresponding binding site to cause an interaction between the scaffold protein and the corresponding binding site.

45. The kit of claim 44 wherein said first and second component are selected from a protein, a small molecule, a fluorophore, a signal peptide, a nanoparticle, a cellular component, and combinations thereof.

46. The method of claim 12 or 34, wherein the effector molecule is a nuclease.

47. The method of claim 46, wherein the nuclease is BfiI.

48. The method of claim 46, wherein the nuclease is BmrI.

49. The method of claim 46, wherein the nuclease is Clo051.

50. The method of claim 46, wherein the nuclease is FokI.

51. A method for modifying a genome of an organism comprising the steps of
a. providing an antibody fragment bound to a first component, wherein the first component is a DNA binding molecule; and
b. providing an epitope tag bound to a second component, wherein the second component is an effector molecule capable of modifying gene expression;
wherein said antibody fragment comprises binding specificity for said epitope tag sufficient to cause an interaction between said antibody fragment and said epitope tag.

52. The method of claim 51, wherein the DNA binding molecule is a DNA, RNA, or protein and the effector molecule is an endonuclease, the interaction of which induces a change in gene expression or a modification of a genomic DNA sequence or base pair.

53. A cell modified according to the method of claim 51 or 52.
